(19) United States
(12) Patent Application Publication
(10) Pub. No.: US 2008/0039864 A1
(43) Pub. Date: Feb. 14, 2008

(54) Cervical tenaculum and methods of use

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSUITE, LLC, San Francisco, CA (US)

(21) Appl. No.: 11/503,369

(22) Filed: Aug. 10, 2006

(51) Int. Cl. A61B 17/42; A61M 29/00

(52) U.S. Cl. 606/119; 606/191

(57) ABSTRACT

A cervical tenaculum is provided having an applicator member with a longitudinal lumen and a tubular member dimensioned to reciprocate within the lumen. The applicator and tubular members are first connected one to the other by engaging protrusions extending from the distal end of the tubular member with matching grooves inside the lumen. After positioning the applicator member in the cervical os of a patient, the protrusions are disengaged from the grooves, and the tubular member is disconnected from the applicator member and removed from the patient. The applicator member instead remains positioned in the cervical os and provides access to the uterine cavity by a clinician. In different embodiments, the applicator member is frustoconical in shape and has one or more ridges disposed on its outer surface, and a control arm is connected to the applicator member for accurate positioning into the cervical os.

### FIELD OF THE INVENTION

The present invention relates to a cervical tenaculum for maintaining the cervical os in a dilated position during a gynecological procedure and for providing a clinician with access to the uterine cavity. More particularly, the present invention relates to a cervical tenaculum that can be inserted in the cervical os easily and that remains anchored without perforating the cervical wall, improving the operating flexibility of the clinician and reducing the discomfort to the patient.

### BACKGROUND OF THE INVENTION

A cervical tenaculum is a gynecological instrument that is used by clinicians to hold the cervix of a patient in a dilated position during a variety of gynecological procedures that require access to the uterus.

One such procedure is endometrial biopsy, which is used to collect cell samples form the uterine wall. A cervical tenaculum is applied to hold the cervix in a steady position, enabling a clinician to insert first a uterine sound through the cervical opening to determine the dimensions and position of the uterus, and successively a catheter to collect small fragments of endometrial tissue by applying aspiration.

Another such procedure is laparoscopy, which is used to examine and treat the pelvic and abdominal organs with a small lighted instrument called a laparoscope. A cervical tenaculum is placed on the cervix of the patient to enable the clinician to determine position and movements of the uterus before an incision is made on the lower abdomen. A laparoscope is then inserted through the incision to examine the abdominal organs and to perform the required surgeries, for example, gallbladder removal or aspiration and excision of ovarian cysts.

Known cervical tenacula are typically shaped like scissors with inwardly-directed spikes at their ends. The gripping action of the tenaculum is achieved by pressing and penetrating the spikes into the cervical tissue, which causes bleeding, trauma and pain for the patient. It is sometimes necessary to use two tenacula, a procedure which not only increases discomfort to the patient, but also causes the activity of the clinician to be more awkward and less flexible. After use, cervical tenacula must be sterilized, because their high unit costs makes it economically unfeasible to dispose of the tenacula after use.

Apparatus have been disclosed in the prior art that teach improvements on the basic tenaculum. For example, in U.S. Pat. No. 5,059,198 to Gimpelson, a cervical tenaculum is disclosed having two end teeth supported by semi-flat surfaces that improve distribution of the gripping force on the cervical tissue. Gimpelson's invention, however, resolves none of the problems of bleeding and discomfort for the patient, of awkwardness during use, and of sterilization costs.

In U.S. Pat. No. 5,499,997 to Sharpe et al., a spring-activated endoscopic tenaculum is disclosed, having sharp-pointed teeth that are positioned to grasp large amounts of tissue. This tenaculum exposes no outer sharp edges after penetration into female genitalia, improving the safety of the tool. Sharpe's invention, however, still does not resolve the traumatic, awkward and costly nature of tenacula in the prior art.

In U.S. Pat. No. 6,773,418 to Sharrow et al, a cup-shaped seal member is disclosed that is positioned around the cervix, and that provides a container for drawing vacuum and increase adhesion to the cervix. This container operates as a conduit for dispensing medications and for introducing instruments into the uterus. While Sharrow's invention reduces some of the most traumatic features of tenacula in the prior art, it is still relatively awkward to use, due to its bulky size, its multiple components and the need for auxiliary systems, such as vacuum.

Therefore, it would be desirable to provide a cervical tenaculum that that is atraumatic to the patient and that does not cause tissue perforations and internal bleedings.

It would also be desirable to provide a cervical tenaculum that is compact in size and that improves operation and flexibility of the clinician.

It would further be desirable to provide a cervical tenaculum that is inexpensive to manufacture and disposable after use.

### SUMMARY OF THE INVENTION

In view of the foregoing, it is an object of the present invention to provide a cervical tenaculum that overcomes the drawbacks of the previously known designs, improving comfort for the patient and operating flexibility for the clinician while reducing unit costs.

It is another object of the present invention to provide a cervical tenaculum that can be employed without causing perforations of the cervical or uterine tissues and the related bleeding.

It also an object of the present invention to provide a cervical tenaculum that is minimally invasive, improving the flexibility of operation of the clinician.

It is a further object of the present invention to provide a cervical tenaculum that can be employed without causing trauma to the patient.

It is still a further object of the present invention to provide a cervical tenaculum that can be manufactured with low cost production methods and that can be disposed after use.

These and other objects of the present invention are accomplished by providing a cervical tenaculum having an applicator member with an inner longitudinal lumen and a tubular member shaped to reciprocate within the lumen. The applicator member and the tubular member are first connected one to the other by engaging protrusions extending from the distal end of the tubular member with matching grooves inside the lumen. After positioning the applicator member in the cervical os of a patient, the protrusions are disengaged from the grooves, and the tubular member is disconnected from the applicator member and removed from the patient. The applicator member instead remains positioned in the cervical os and provides access to the uterine cavity through the inner lumen.

In one embodiment, the applicator member is frustoconical in shape and has a plurality of grooves disposed on its outer surface, which engage the walls of the cervical os in a screw-like fashion that anchors the applicator member to the os. In another embodiment, the positioning of the applicator member in the cervical os is aided by a control arm that is connected to the applicator member and that is actuated by the clinician.

The ridges on the applicator member may be arranged in a variety of patterns, for example, in a spiral pattern, or in a circular pattern essentially perpendicular to the longitudinal axis of the applicator member, or as discreet segments distributed on its outer surface.

The applicator member is typically made of a resilient material, such as a silicone material, providing a tenaculum that retains its shape during use but that is not excessively harsh on the neighboring tissue, as in the case of a metal tenaculum. The use of a plastic material also provides for lower unit costs by allowing mass manufacturing with industrial processes such as injection molding.

Typically, the tenaculum according to the present invention has two grooves carved in the lumen of the applicator member and two protrusions extending from the tubular member. Each of the two grooves and two protrusions is spaced from the other both angularly (for example, by 90 or 180 degrees), and also longitudinally along the axes of the applicator and tubular members. One skilled in the art will recognize that different numbers and dispositions of the groves and protrusions are possible and still within the scope of the present invention.

An optional control arm is connected to the applicator member, by having, in one embodiment, a hook-shaped distal end engage a loop attached to the applicator member. The control arm enables the clinician to maneuver the applicator member during insertion and find the most appropriate anchoring to the cervical os.

Methods for using the cervical tenaculum according to the present invention are also disclosed herein.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is an exploded view of a first embodiment of the present invention, wherein the applicator member is illustrated in a cross-sectional view;

FIG. 2 is a bottom view of the applicator member, showing the proximal base, the lumen, and the grooves therein;

FIGS. 3A-3C are top views of different embodiments of the tubular member;

FIG. 4 is a side view of the applicator member with ridges disposed the lateral wall;

FIGS. 5A-5C are side views of alternative embodiments of the applicator member, each having a different disposition of the ridges disposed on the lateral wall;

FIGS. 6A-6C are detail views of alternate configurations of the ridges on the applicator member;

FIG. 7 is a side view of an embodiment of the cervical tenaculum having a control arm; and

FIG. 8 is a schematic view of a cervical tenaculum inserted into the cervical os.

### DETAILED DESCRIPTION OF THE INVENTION

The present invention is directed to a cervical tenaculum having an applicator member with a longitudinal lumen and a tubular member shaped to reciprocate within the lumen. The applicator member and the tubular member are first connected one to the other by engaging protrusions extending from the distal end of the tubular member with matching grooves inside the lumen. After positioning the applicator member in the cervical os of a patient, the protrusions are disengaged from the grooves, and the tubular member is disconnected from the applicator member. The applicator member instead remains positioned in the cervical os and provides access to the uterine cavity by a clinician.

In one embodiment, the applicator member is frustoconical in shape and has grooves disposed on its outer surface, engaging and anchoring the applicator member to the walls of the cervical os in a screw-like fashion.

In another embodiment, the positioning of the applicator member in the cervical os is aided by a control arm that is connected to the applicator member and that is actuated by the clinician.

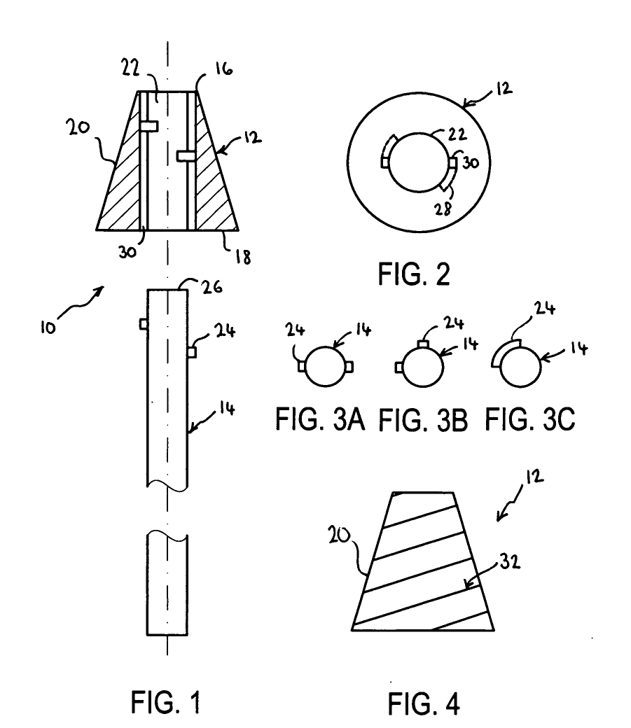

Referring to FIGS. 1 and 2, exemplary embodiment 10 of a cervical tenaculum constructed in accordance with the principles of the present invention is described. Cervical tenaculum 10 includes an applicator member 12 (shown in a cross-sectional view) and a tubular member 14 shaped to engage and disengage one from the other. Applicator member 12 has a frustoconical shape, comprising distal base 16, proximal base 18, and lateral wall 20 that connects distal base 16 with proximal base 18. Lumen 22 extends longitudinally through applicator member 12 in a longitudinal direction, connecting an opening in distal base 16 with an opening in distal base 18 and providing applicator member 12 with a ring-like structure.

Applicator 12 is typically made of a resilient material, such as a silicone material, that retains its basic configuration while providing for a relatively soft contact with the os of the patient. Further, when applicator member 12 is made from a silicone material, an injection molding process can be employed, reducing the manufacturing costs of the tenaculum in comparison with the metal tenacula in the prior art, and providing for a tenaculum that can be disposed after use, thereby avoiding the cleaning and sterilization processes required by tenacula in the prior art.

Applicator member 12 is dimensioned to comfortably fit into the cervical os, and typically has a distal base with a diameter of approximately 6 mm, a proximal base with a diameter of approximately 14 mm, and a lateral wall that is approximately 14 mm long. One skilled in the art will recognize that other dimensions may be used, to fit patients of different sizes.

Tubular member 14 is generally cylindrical in shape, and has an outer diameter that allows tubular member 14 to reciprocate within lumen 22. A plurality of protrusions 24 extend radially in the proximity of distal end 26 of tubular member 14. A plurality of corresponding lateral grooves 28 are carved angularly within lumen 22, with dimensions and positions that correspond to the dimensions and positions of protrusions 24. Consequently, when distal end 26 is inserted within lumen 22, protrusions 24 first travel along longitudinal grooves 30 until an alignment with lateral grooves 28 is achieved, and successively, upon an angular rotation of tubular member 14, protrusions 24 engage lateral grooves 28, creating a stable connection between applicator member 12 and tubular member 14.

One skilled in the art will recognize that protrusions 24 may have different shapes, for instance, cylindrical shapes with sharp or rounded edges, or semi-spherical shapes, and that longitudinal grooves 30 and lateral grooves 20 may be produced with corresponding shapes. Further, protrusions 24 may be positioned at the same distance from distal end 26, or may be longitudinally spaced from one another. Still further, as illustrated in FIGS. 3A-3C, protrusions 24 may be positioned at different angles from one another, for instance, at 90 or 180 degrees from one another, as in FIGS. 3A and 3B, or a single protrusion may be present, as in FIG. 3C, or more than two protrusions.

One skilled in the art will also recognize that different arrangements of lateral grooves 28 and protrusions 24 are possible. For example, in one embodiment, lateral grooves 28 may be disposed in a direction that is not perpendicular to the longitudinal axis of applicator member 12; in particular, lateral grooves 28 may be disposed in a spiral pattern inside lumen 22, and protrusion 24 may engage lateral grooves 28 after tubular member 14 is rotated inside lumen 22 with a twisting motion. In another embodiment (not shown), tubular member 14 may have a threaded distal end 26 that engages a corresponding threaded pattern in lumen 22. In this embodiment, applicator member 12 and tubular member 14 are joined and separated in the same manner as a nut with a bolt.

Tubular member 14 may have a solid cross-section, like a tubular rod, or a hollow center, like a tubular conduit. While a solid cross-section provides for greater rigidity, a hollow cross-section will enable a clinician to use tubular member 14 as conduit for inserting other instruments, like a catheter.

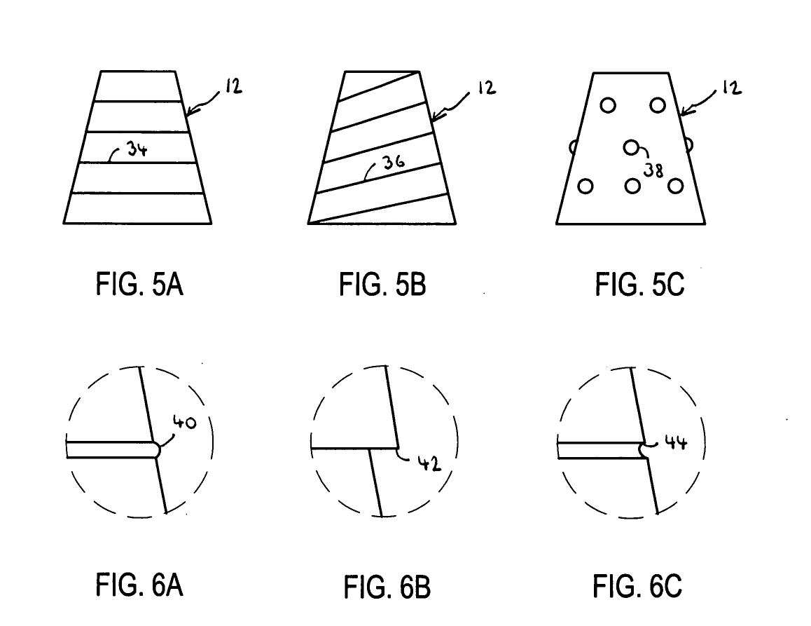

Referring now to FIG. 4, one or more ridges 32 may disposed on outer surface 20 of applicator member 12, and are shaped to engage and provide a stable anchoring of applicator member 12 to the cervical os. As shown in FIGS. 5A-5C, ridges 32 may be disposed in parallel circular pattern 34 perpendicular to the longitudinal axis of applicator member 12, or in spiral pattern 36, or in discrete segments, exemplified in FIG. 5C as a plurality of semi-spherical segments 38.

Ridges 32 may also have different contours. For example, as shown in FIGS. 6A-6C, ridges 32 may have rounded contour 40, or scalloped contour 42 defined by layers of different height on lateral wall 20, or be formed as grooves 44 on lateral wall 20. One skilled in the art will appreciate that different contours, densities and dispositions of ridges 32 are possible and still within the spirit of the present invention.

In the preferred embodiment, grooves 44 are disposed in a spiral pattern on outer surface 20, and are defined by grooves 44, providing applicator 12 with a screw-like shape that adheres to the inner cervical walls.

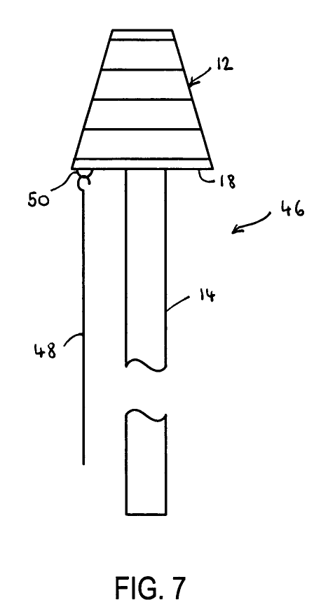

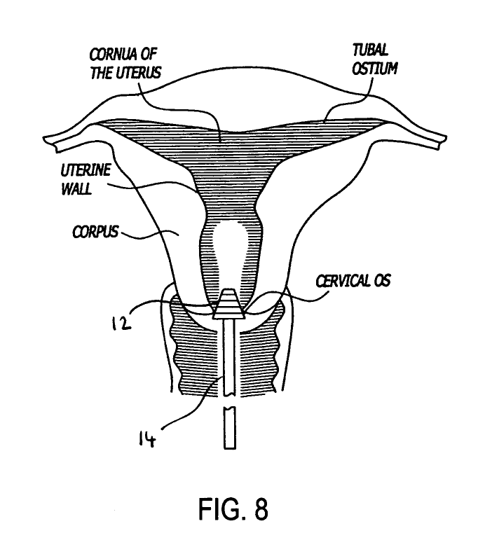

Referring now to FIG. 7, another embodiment 46 of the invention includes the application of control arm 48 to applicator member 12, that may be rigid or semi-rigid, and that facilitates an accurate positioning of applicator member 12 into the patient's cervical os. Control arm 48 is preferably connected to proximal base 18, to provide the clinician with visual guidance and to have control arm 48 in the least intrusive position during insertion. Control arm 48 and proximal base 18 may be connected in a variety of ways, for example, by having a hook attached to the distal end of control arm 48 anchored in loop 50 extending from proximal base 18.

A method for using the above-described cervical tenaculum is described hereinafter. In a first step, applicator member 12 and tubular member 14 are connected one to the other by inserting distal end 26 of tubular member 14 into lumen 22 of applicator member 12, and by matching protrusions 24 with grooves 28. The clinician may be alerted that protrusions and grooves are in matching positions in a variety of ways, for example, by having protrusions 24 and grooves 28 match when the distal end 26 reaches distal base 16, or by having markings disposed on tubular member 14 indicate the requited depth of insertion into lumen 22. Alternatively, applicator member 12 and tubular member 14 may be supplied to the clinician in pre-assembled condition.

In a second step, the clinician rotates the tubular member 14, locking protrusions 24 in grooves 28, and achieving a stable connection between applicator member 12 and tubular member 14, so that a longitudinal translation of tubular member 14 will cause an equal translation of applicator member 12.

In a successive step, the clinician inserts the cervical tenaculum in the vaginal canal of the patient until the applicator member contacts the cervical os. If necessary, the clinician will twirl applicator member 12 by moving tubular member 14 and, if provided, by acting on control arm 48, until applicator member 12 is securely positioned in the os. Ridges 32 on applicator member 12 provide an anchoring action, preventing an undesired release of applicator member 12 due to the constrictive action of the os and movements of the patient's body.

In the next step, the clinician counter-rotates tubular member 14, disengaging protrusions 24 from grooves 28, and extracts tubular member 14 from the patient's body, while leaving applicator member 12 positioned in the cervical os. Applicator member 12 maintains then the os in a dilated position in and enables access to the uterus through the lumen of the applicator member.

While preferred embodiments of the invention are described above, it will be apparent to one skilled in the art that various changes and modifications may be made. The appended claims are intended to cover all such changes and modifications that fall within the spirit and scope of the invention.

What is claimed is:

1. A cervical tenaculum comprising:
an applicator member having a distal opening, a proximal opening, and a longitudinal lumen therebetween, the longitudinal lumen comprising one or more depressions extending laterally from the lumen; and
a tubular member having an outer diameter dimensioned for reciprocating within the lumen, the tubular member comprising one or more protrusions extending laterally from the distal end of the tubular member, the protrusions being positioned on the tubular member to mate with the depressions,
wherein an insertion and rotation of the tubular member within the lumen causes the protrusions to engage the depressions, thereby causing the tubular member to connect with the applicator member, and further causing the applicator member to translate upon a translation of the tubular member, and
wherein a counter-rotation of the tubular member causes the protrusions to disengage from the depressions and the tubular member to disconnect form the applicator member.

2. The cervical tenaculum of claim 1, wherein the applicator member has a frustoconical shape including a smaller base and a larger base connected by a lateral wall, wherein the distal opening is situated in the smaller base, and wherein the proximal opening is situated in the larger base.

3. The cervical tenaculum of claim 1, wherein the applicator member is dimensioned to fit in the cervical os of a patient.

4. The cervical tenaculum of claim 1, wherein the applicator member comprises one or more ridges on its outer surface.

5. The cervical tenaculum of claim 4, wherein the one or more ridges are defined by grooves carved on the outer surface.

6. The cervical tenaculum of claim 4, wherein the one or more ridges are defined by protrusions extending from the outer surface.

7. The cervical tenaculum of claim 4, wherein the one or more ridges are defined by layers of varying heights disposed on the outer surface.

8. The cervical tenaculum of claim 4, wherein the one or more ridges are disposed in a spiral pattern.

9. The cervical tenaculum of claim 8, wherein the ridges are defined by grooves disposed in the spiral pattern on the outer surface to provide a screw-shaped pattern.

10. The cervical tenaculum of claim 4, wherein the one or more ridges are disposed in a circular pattern essentially perpendicular to the lumen.

11. The cervical tenaculum of claim 1, wherein the applicator member is made of a resilient material.

12. The cervical tenaculum of claim 11, wherein the resilient material is a silicone material.

13. The cervical tenaculum of claim 1, wherein there are two depressions angularly spaced from each other, and wherein there are two protrusions.

14. The cervical tenaculum of claim 13, wherein the two depressions are longitudinally spaced along the lumen, and wherein the two protrusions are longitudinally spaced along the tubular member in corresponding positions.

15. The cervical tenaculum of claim 13, wherein the two protrusions are angularly spaced by approximately 180 degrees.

16. The cervical tenaculum of claim 1, wherein the one or more protrusions are disposed to form a first spirally-wound threaded pattern, and wherein the one or more depressions are disposed to form a second spirally-wound threaded pattern shaped to mate with the first spirally-wound threaded pattern.

17. The cervical tenaculum of claim 1, further comprising a control arm connected to the applicator member.

18. The cervical tenaculum of claim 16, wherein the applicator member has a frustoconical shape, and wherein the control arm is connected to the larger base of the applicator.

19. The cervical tenaculum of claim 16, wherein the control arm comprises an elongated member having a hook-shaped distal end, and wherein the control arm is connected to the applicator member by having the hook-shaped distal end engage a loop extending from the applicator member.

20. A method for applying a cervical tenaculum on a patient, the method comprising:
providing the cervical tenaculum, wherein the cervical tenaculum includes an applicator member having a longitudinal lumen having one or more depressions extending laterally from the lumen and a tubular member having an outer diameter dimensioned for reciprocating inside the lumen, the tubular member further having one or more protrusions extending laterally and positioned to mate with the depressions;
inserting and rotating the tubular member within the lumen, causing the protrusions to engage the depressions, further causing the tubular member to connect with the applicator member, the applicator member translating upon a translation of the tubular member;
positioning the applicator member in the cervical os of the patient by translating the tubular member into the patient's vaginal canal; and
counter-rotating, disengaging and retracting the tubular member from the applicator member, thereby causing the tubular member to disconnect from the applicator member.

21. The method of claim 20, further comprising the step of anchoring the applicator member in the cervical os after positioning in the cervical os by providing ridges on the outer surface of the applicator member.

22. The method of claim 21, wherein the ridges are disposed in a spiral patterns.

23. The method of claim 20, wherein the tubular member is provided with the one or more protrusions disposed to form a first spirally-wound threaded pattern, and wherein the applicator is provided with the one or more depressions disposed to form a second spirally-wound threaded pattern shaped to mate with the first threaded pattern.

24. The method of claim 20, further comprising the step of controlling the positioning of the applicator member by providing a control arm connected to the applicator member.
